(19) United States
(12) Patent Application Publication
(10) Pub. No.: US 2006/0269509 A1
(43) Pub. Date: Nov. 30, 2006

(54) Scent eliminating composition including colloidal silver

(75) Inventors: Gregory Alan Gumbrecht, Davisburg, MI (US); Craig Allen Stawiarski, Farmington Hills, MI (US); Jeffrey Scott Stawiarski, Brighton, MI (US)

Correspondence Address: BUTZEL LONG;DOCKETING DEPARTMENT, 100 BLOOMFIELD HILLS PARKWAY, SUITE 200, BLOOMFIELD HILLS, MI, 48304, US

(73) Assignee: Excelda Corporation

(21) Appl. No.: 11/141,809

(22) Filed: May 31, 2005

(51) Int. Cl. A61L 9/00; A61K 33/38

(52) U.S. Cl. 424/76.2; 424/618

(57) ABSTRACT

A scent eliminating composition in accordance with the present invention comprises colloidal silver, a morpholinium alkyl sulfate, and a liquid or gelatinous base. Preferably, the colloidal silver is provided in the form of silver colloidal water, which consists of tiny metallic silver particles suspended in deionized water. A preferred morpholinium alkyl sulfate is soyaethyl morpholinium ethosulfate.

### BACKGROUND OF THE INVENTION

The present invention relates generally to deodorizing and scent eliminating sprays and, in particular, to a scent eliminating composition including colloidal silver.

Deodorants and scent eliminating sprays are well known. Such deodorants and sprays have often been used, for example, by hunters in order to avoid scent detection by his or her prey. Some prior art scent eliminators merely cover or mask a hunter's scent, which allows prey to still detect a scent. A common ingredient found in these scent eliminators is sodium bicarbonate or baking soda, which can disadvantageously leave a white powdery residue on the user's clothing when applied. Other prior art scent eliminators utilize carbon as a base ingredient, which can disadvantageously stain clothing or bleed or fade clothing colors when is applied. In addition, regardless of the base ingredient, many of these prior art scent eliminators fight or suppress odors for only a short period of time.

Soyaethyl morpholinium ethosulfate is well known and has been utilized in prior art scent eliminating solutions. This material is typically liquid and advantageously does not stain or discolor clothing or leave any residue when applied. This material, however, works only on the vapor phase of the odor and, therefore, works for a predetermined period of time (although for a greater period of time than either baking soda-based or carbon-based scent eliminators) based on the amount of vapors present.

It remains desirable to provide a scent eliminator that may be applied to clothing without damaging the clothing or leaving any residue on the fabric of the clothing while also providing scent detection protection for an extended period of time.

### SUMMARY OF THE INVENTION

The present invention concerns a scent eliminating composition. The composition comprises: colloidal silver, a morpholinium alkyl sulfate, and a liquid or gelatinous base.

The invention also concerns a method of preparing a scent eliminating composition. The method comprises the steps of: a) providing a supply of colloidal silver; b) providing a supply of a morpholinium alkyl sulfate; c) providing a supply of a liquid or gelatinous base; and d) mixing together the colloidal silver, the morpholinium alkyl sulfate, and the base in a manner effective to disperse the morpholinium alkyl sulfate throughout the base and to suspend the colloidal silver in the base.

The invention further concerns a method of eliminating a scent, comprising applying to a locus in need of such scent elimination an effective amount of a composition comprising: colloidal silver, a morpholinium alkyl sulfate, and a liquid or gelatinous base.

### DESCRIPTION OF THE PREFERRED EMBODIMENT

The scent eliminating composition in accordance with the present invention comprises colloidal silver, a morpholinium alkyl sulfate, and a liquid or gelatinous base. Preferably, the colloidal silver is provided in the form of silver colloidal water, which consists of tiny metallic silver particles suspended in deionized water. A preferred morpholinium alkyl sulfate is soyaethyl morpholinium ethosulfate.

These ingredients can be combined in any suitable amounts effective to eliminate the applicable scent(s). Preferably, the composition comprises from about 8 ppm to about 16 ppm colloidal silver, from about 0.25 wt % to about 1.0 wt % morpholinium alkyl sulfate, and from about 99.0 wt % to about 99.75 wt % base; and more preferably from about 5 ppm to about 20 ppm colloidal silver, from about 0.10 wt % to about 1.25 wt % morpholinium alkyl sulfate, and from about 98.75wt % to about 99.9 wt % base.

When the colloidal silver is provided in the form of silver colloidal water, the composition preferably comprises from about 1.5 wt % to about 3.75 wt % of 500 ppm silver colloidal water, from about 0.15 wt % to about 1.0 wt % morpholinium alkyl sulfate, and from about 95.25 wt % to about 98.25 wt % additional deionized water, by weight of the composition

A typical composition according to the invention contains the following amounts of ingredients:

| % by Weight | Ingredient |
|---|---|
| 96.98% | deionized water |
| 0.52% | soyaethyl morpholinium ethosulfate |
| 2.5% | silver colloidal water (500 ppm) |

While the above percentages by weight have been described to be an embodiment of the invention, those skilled in the art will appreciate that the percentages may vary while remaining within the scope of the invention.

The colloidal silver consists of tiny particles of silver metal, not silver ions. Preferably, the size of the silver particles is about 10 microns (micrometers) or smaller, and typically between about 1 millimicron (1 nanometer) and about 1 micron. The silver particles are suspended in a liquid or gelatinous base, as described below. The colloidal silver can be provided in the composition in any suitable form. Preferably, the colloidal silver is provided in the form of silver colloidal water, which consists of tiny metallic silver particles suspended in deionized water. Preferably, the concentration of the metallic silver particles in the silver colloidal water is from about 475 ppm to about 525 ppm, and typically about 500 ppm. Silver colloidal water is available from numerous manufacturers, such as, but not limited to, Invive.

The metallic silver particles can be suspended in the base by any suitable means. In some cases, the very small size of the particles, and the resulting large surface area to volume ratio, is sufficient by itself to keep the particles suspended in the base. In silver colloidal water, the silver particles are usually provided with a small electrical charge to help keep them suspended in the deionized water. Alternatively, a stabilizer can be added to the composition to keep the particles suspended.

The base can be any liquid or gelatinous material suitable for suspending the colloidal silver and dispersing the morpholinium alkyl sulfate, and suitable for providing with the other ingredients a scent eliminating composition having the desired properties and efficacy. For example, in a preferred embodiment, the scent eliminating composition is formulated as a sprayable composition. Preferably, the base is deionized water; i.e., water from which both anions and cations have been removed by an ion exchange process. The use of deionized water is advantageous when the silver particles have been charged to keep them suspended. Alternatively, the base can be distilled water; i.e., water that has been purified by passing through an evaporation-condensation cycle. Some nonlimiting examples of other possible bases include siliconized water, various natural oils, and various cream, lotion and gel bases.

The morpholinium alkyl sulfates have been known for some time for use in controlling odors. They can be made by any suitable process, such as any of those described in U.S. Pat. Nos. 2,417,992, 2,852,434 and 4,851,214. The material is preferably an alkyl morpholinium alkyl sulfate, and more preferably an N-alkyl-N-ethylmorpholinium ethyl sulfate. In a particularly preferred embodiment, the material is a soyaethyl morpholinium ethosulfate. U.S. Pat. No. 4,851,214 describes one suitable process for making this material.

Soyaethyl morpholinium ethosulfate may be obtained, for example, under the trade name of Forestall™ offered for sale by the Uniqema Corporation, or from similar material suppliers. Uniqema describes the soyaethyl morpholinium ethosulfate as a cationic surfactant that is proven to neutralize a wide range of malodors, by complexing specific organic molecules, particularly mercaptan and sulfur based odors, and reducing their concentration in the vapor phase.

The scent eliminating composition of the invention can be prepared by any suitable method. The colloidal silver, morpholinium alkyl sulfate, and base are mixed together in a manner effective to disperse the morpholinium alkyl sulfate throughout the base and to suspend the colloidal silver in the base. Any suitable mixing conditions can be used. For example, a composition containing silver colloidal water, additional deionized water, and soyaethyl morpholinium ethosulfate may be mixed well using a high speed rotary mixer, at room temperature, for a time between about 15 minutes and about 30 minutes, e.g., about 20 minutes.

The ingredients can usually be mixed in any suitable order. When the composition includes silver colloidal water and additional deionized water, it is preferred to first mix these ingredients together and then mix in the morpholinium alkyl sulfate.

The scent eliminating composition in accordance with the present invention is preferably packaged in a spray device (not shown). The spray device is preferably a mechanical (i.e. non-aerosol propellant) spray device and may be any commercially available spray device including, but not limited to, mixor type models available from Calmar. that are operable to emit the scent eliminating composition in accordance with the present invention in a mist or to atomize the scent eliminating solution.

When the scent eliminating composition in accordance with the present invention is applied to a user, the morpholinium alkyl sulfate part of the scent eliminating solution works in the vapor phase of the odor by complexing or neutralizing the odor. Importantly, the source of the odor does not need to be from a bacterial source for the morpholinium alkyl sulfate to be effective. Morpholinium alkyl sulfate is effective on the odors of tobacco smoke, fish odors, leather, etc. Morpholinium alkyl sulfate is also effective on organic acids that are found in body odor.

While the morpholinium alkyl sulfate is complexing or neutralizing the vapor phase of the odor(s), the silver particles are eliminating the bacteria that causes body odor. The silver particles work directly on bacteria, destroying the bacteria before it has a chance to break down and cause the body odor.

The morpholinium alkyl sulfate works by reducing the vapor phase of the odor or, more specifically, by reducing the effect of the odor. The silver particles in the composition, in contrast, work on the source of the odor, reducing the likelihood that the source of the odors will produce more odors, which results in an increased length of time that the scent eliminating solution in accordance with the present invention is effective. The morpholinium alkyl sulfate and the colloidal silver, therefore, work in conjunction to eliminate both existing scents or odors and the potential source of the scents or odors when the scent eliminating solution in accordance with the present invention is applied. Because of this, the scent eliminating composition in accordance with the present invention is effective for a longer period of time than the scent eliminators of the prior art such as baking soda-based scent eliminators, carbon-based scent eliminators, or those scent eliminators having only soyaethyl morpholinium ethosulfate as an ingredient.

When applied, the scent eliminating composition in accordance with the present invention will advantageously not stain clothing or bleed or fade clothing colors, like the carbon-based scent eliminators of the prior art. The scent eliminating composition in accordance with the present invention will also not leave a white powdery residue, like the baking-powder-based scent eliminators of the prior art. In testing, the liquid compound in accordance with the present invention is long-lasting as well as cost-effective to produce and package.

The scent eliminating composition in accordance with the present invention may be advantageously used for eliminating odors, removing odors, neutralizing odors, and complexing odors, such as for a hunter that wishes to avoid scent detection by his or her prey. More generally, the composition can be applied to any locus in need of scent elimination. For example, the locus may be a human body and/or clothing (including footwear such as boots or shoes), a weapon in the case of a hunter, or a fishing lure in the case of a fisherman.

In accordance with the provisions of the patent statutes, the present invention has been described in what is considered to represent its preferred embodiment. However, it should be noted that the invention can be practiced otherwise than as specifically illustrated and described without departing from its spirit or scope.

What is claimed is:

1. A scent eliminating composition, comprising:
colloidal silver;
a morpholinium alkyl sulfate; and
a liquid or gelatinous base.

2. The composition according to claim 1 wherein said base comprises deionized water.

3. The composition according to claim 1 wherein said morpholinium alkyl sulfate comprises soyaethyl morpholinium ethosulfate.

4. The composition according to claim 1 wherein said composition comprises from about 5 ppm to about 20 ppm colloidal silver, from about 0.10 wt % to about 1.25 wt % morpholinium alkyl sulfate, and from about 98.75 wt % to about 99.9 wt % base.

5. The composition according to claim 1 wherein said colloidal silver is included in the composition by providing silver colloidal water.

6. The composition according to claim 1 wherein said composition is sprayable.

7. The composition according to claim 1 wherein said colloidal silver has a particle size of not greater than about 10 microns.

8. A method of preparing a scent eliminating composition, comprising the steps of:
a) providing a supply of colloidal silver;
b) providing a supply of a morpholinium alkyl sulfate;
c) providing a supply of a liquid or gelatinous base; and
d) mixing together said colloidal silver, morpholinium alkyl sulfate, and base in a manner effective to disperse said morpholinium alkyl sulfate throughout said base and to suspend said colloidal silver in said base.

9. The method according to claim 8 wherein said step c) comprises providing deionized water.

10. The method according to claim 9 wherein said step d) comprises first mixing said colloidal silver and said deionized water, and then mixing said morpholinium alkyl sulfate with said mixture of colloidal silver and deionized water.

11. The method according to claim 8 wherein said morpholinium alkyl sulfate, said base, and said colloidal silver are mixed together for a time of from about 15 minutes to about 30 minutes.

12. The method according to claim 8 wherein said step a) comprises providing a supply of silver colloidal water.

13. A method of eliminating a scent, comprising applying to a locus in need of such scent elimination an effective amount of a composition comprising:
colloidal silver;
a morpholinium alkyl sulfate; and
a liquid or gelatinous base.

14. The method according to claim 13 wherein said composition is applied to at least one of a human body and clothing to eliminate the scent.

15. The method according to claim 13 wherein said composition is applied by spraying it onto said locus.

16. The method according to claim 13 wherein said morpholinium alkyl sulfate comprises soyaethyl morpholinium ethosulfate.

17. The method according to claim 13 wherein said base comprises deionized water.

18. The method according to claim 13 wherein said colloidal silver is included in the composition by providing silver colloidal water.
